(19) United States
(12) Patent Application Publication
(10) Pub. No.: US 2008/0228282 A1
(43) Pub. Date: Sep. 18, 2008

(54) Artificial hip with grooved ball or cup for improved lubrication by capillary action

(76) Inventor: John Brodowski, Smithtown, NY (US)

Correspondence Address: JOHN BRODOWSKI, 81 MOBREY LANE, SMITHTOWN, NY, 11787, US

(21) Appl. No.: 11/717,243

(22) Filed: Mar. 14, 2007

(51) Int. Cl. A61F 2/32

(52) U.S. Cl. 623/22.11; 623/22.15

(57) ABSTRACT

Prosthetic hips are well known in the arts and have been successfully used for many years. There are, however, some inherent health problems associated with present art designs. All of the prosthetic hip designs utilize a variety of materials. Most used are plastic, ceramic and metal materials. One of the main problems associated with prior art prosthetic hips involves friction generated between cup and ball. Friction dislodges, from cup and ball surfaces, plastic, ceramic or metallic fragments during a patient's normal motor functions. These particles interact with the body's chemistry and may cause deleterious health side effects. Rubbing action between cup and ball also produces ions which can adversely affect the patient's health. It is therefore essential that friction between prosthetic hip cup and ball is reduced to a minimum. The design of the prosthetic hip presented herewith will alleviate some of the unwanted friction between cup and ball.

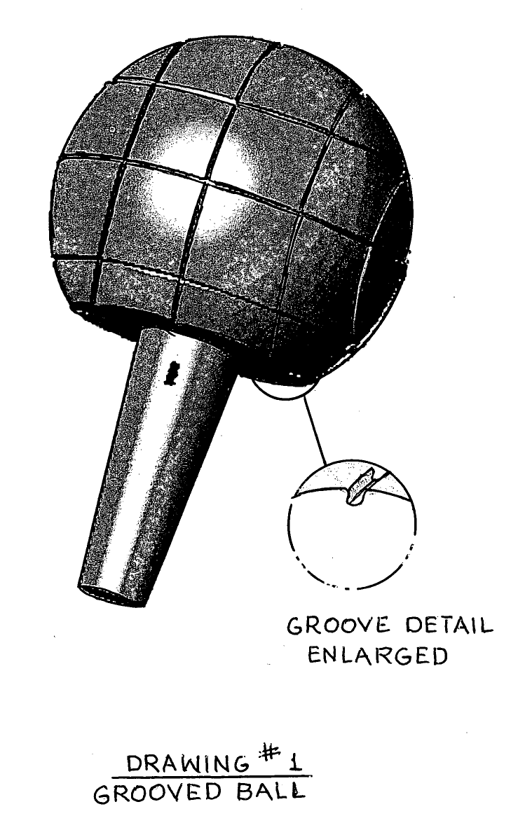

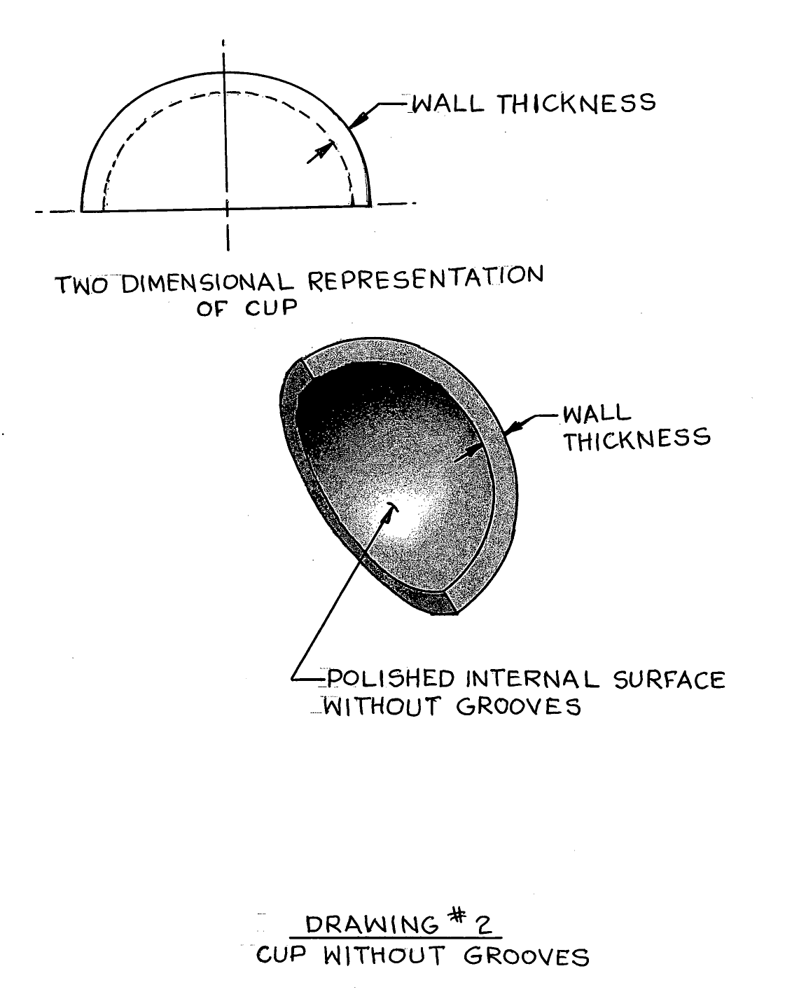

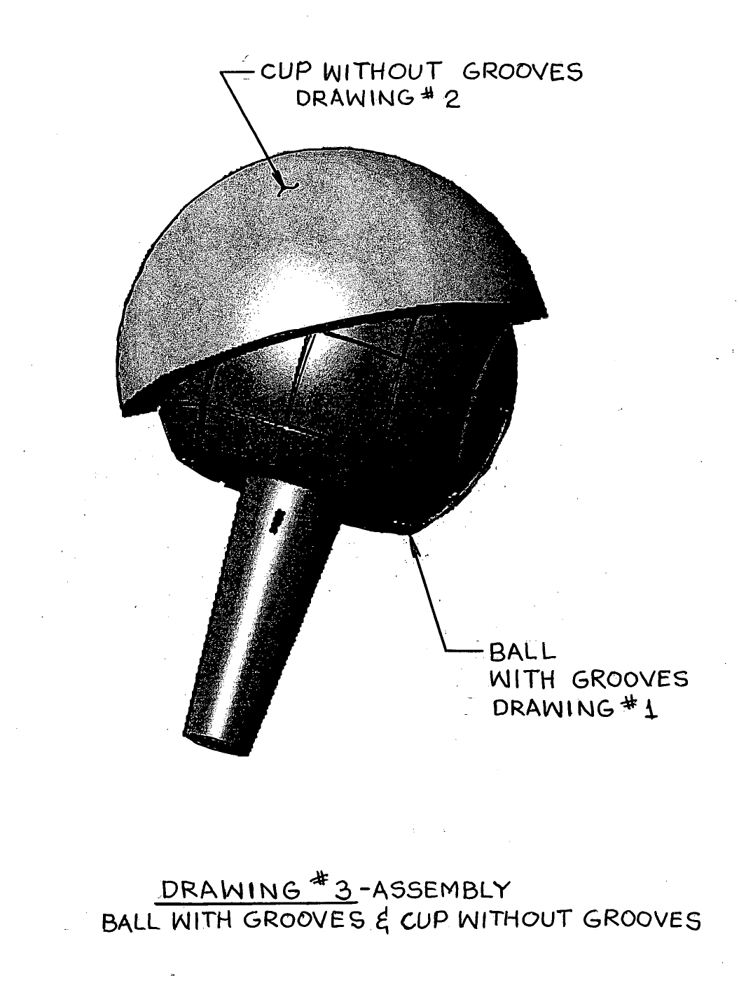

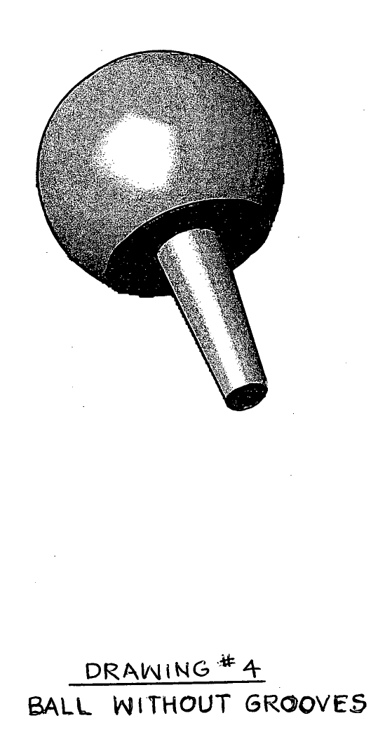

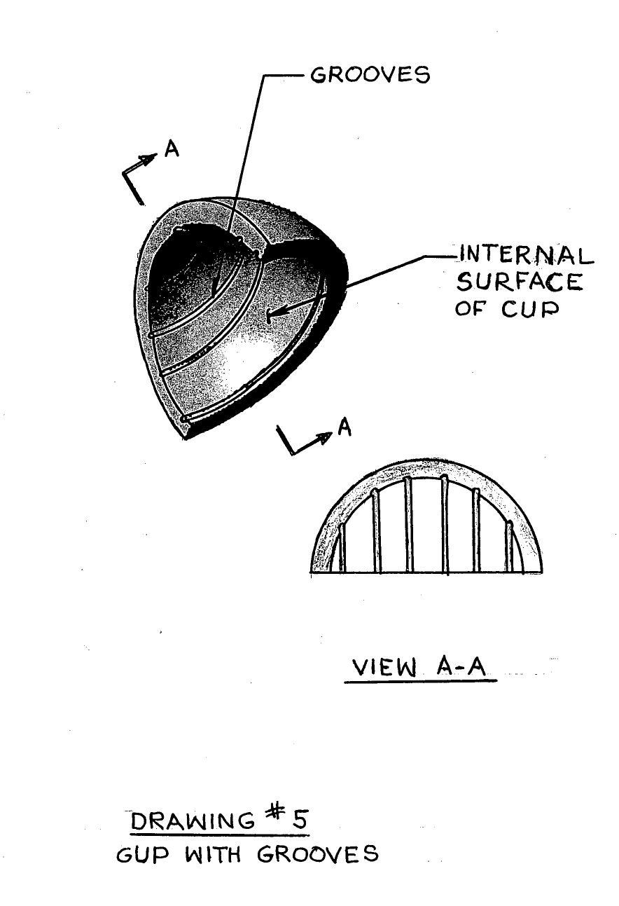

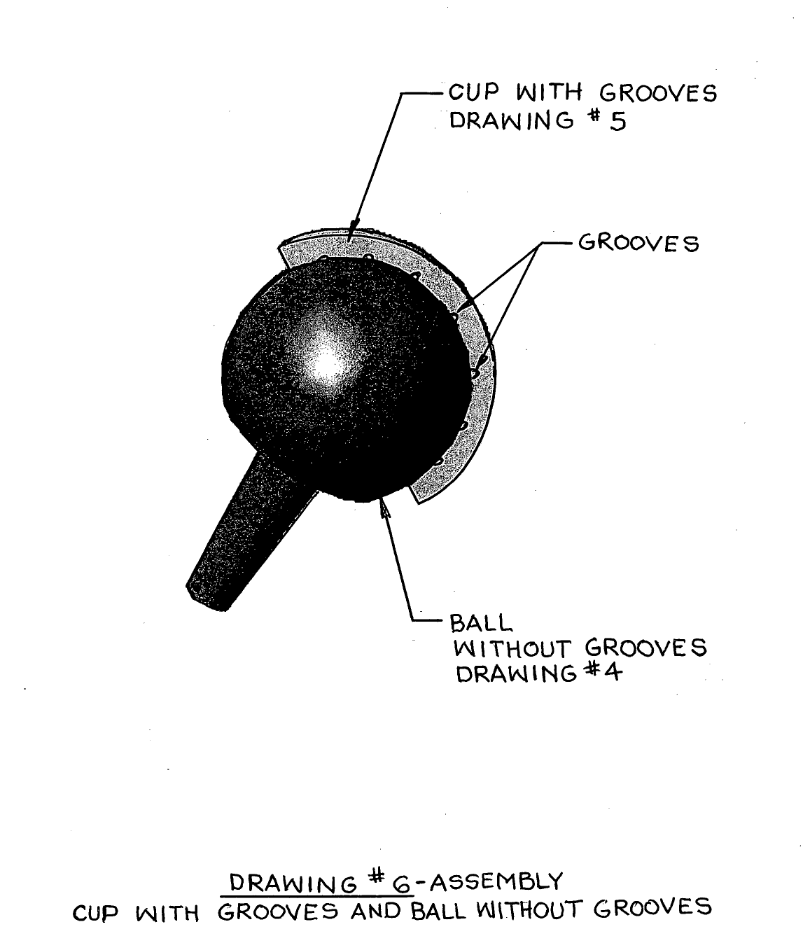

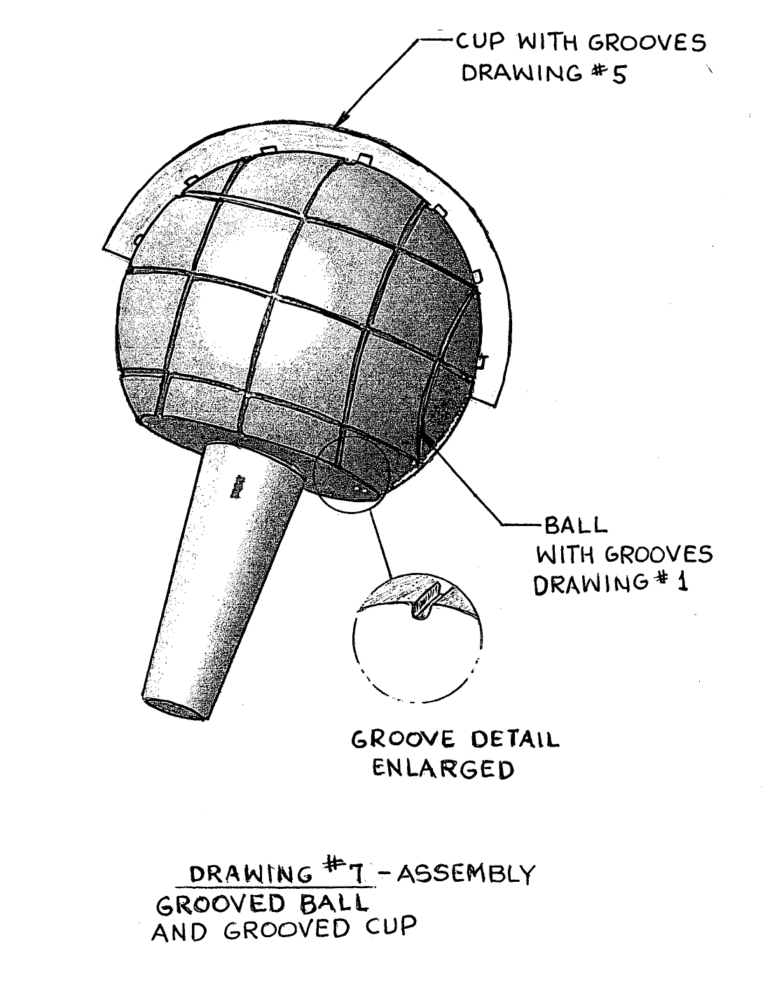

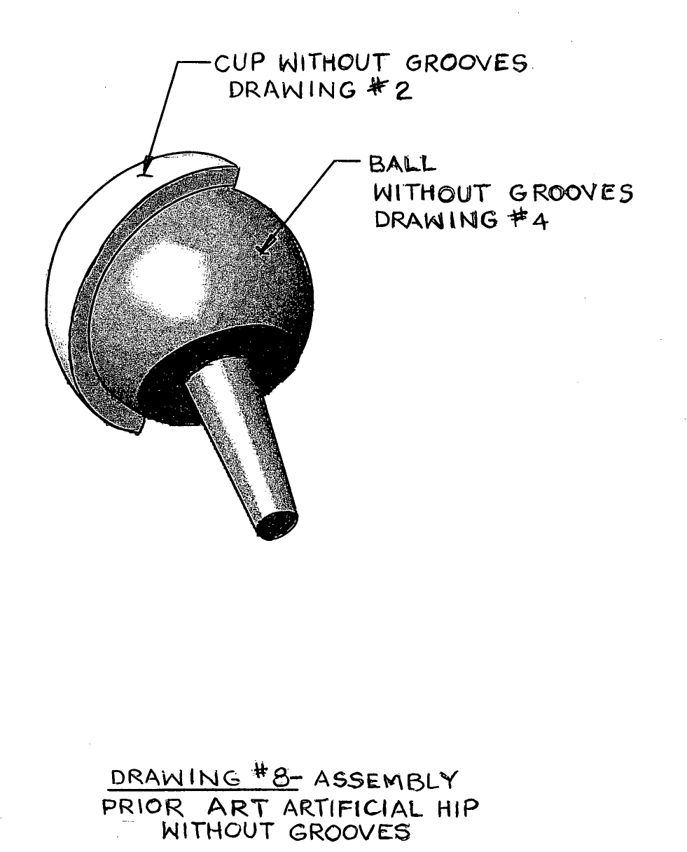

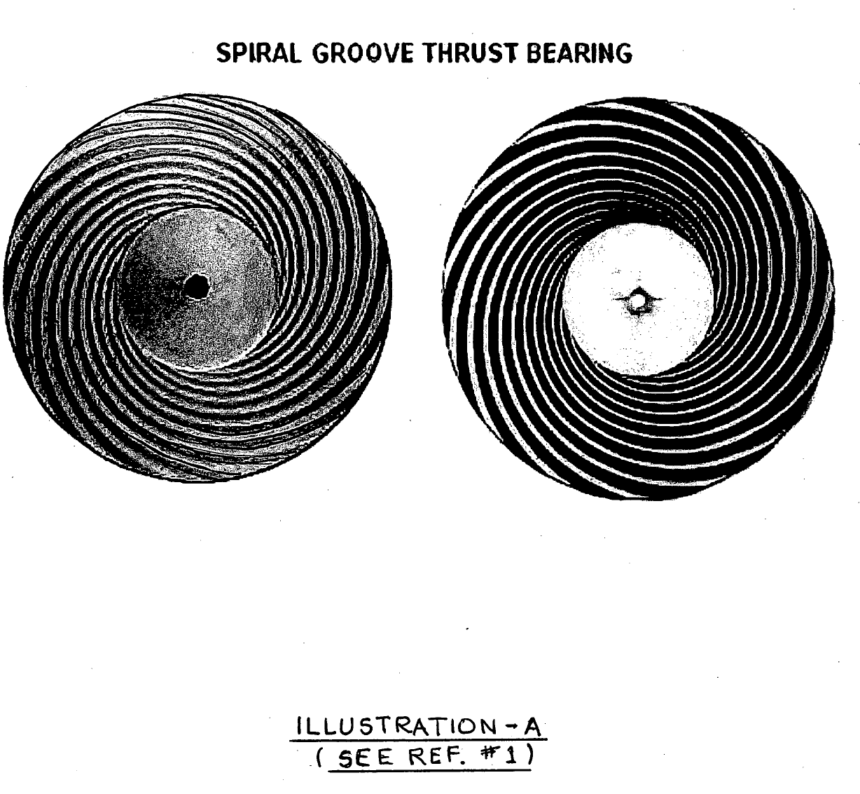

A method of improving prosthetic hip lubrication is presented in this application. This method incorporates a grooved ball or grooved cup in the prior art prosthetic hip.

Three distinct variations of grooved ball and grooved cup are presented which will improve prosthetic hip lubrication.

Variation number one is shown on Drawing #3. This variation is shown with a grooved ball, Drawing #1, and a cup without grooves, Drawing #2.

Variation number two is shown on Drawing #6. This variation is shown with a ball without grooves, Drawing #4, and a cup with grooves, Drawing #5.

Variation number three is shown on Drawing #7. This variation is shown with a ball with grooves, Drawing #1, and a cup with grooves, Drawing #5.

As the patient flexes his hip, the ball is rotated in the cup and through capillary action, or through pumping action caused by the ball's movement, body fluid in time will penetrate all the grooves and will lubricate the rubbing surfaces between the cup and ball.

The size, shape, number, orientation and grooves' finish must be determined experimentally for optimum lubricating action of the grooves.

Many types of present art bearings routinely utilize grooves in their designs in order to improve lubrication and thus reduce friction and improve performance of the bearings.

A proven principle of improved bearing lubrication utilized in the bearing industry is incorporated in this prosthetic hip design.

### PRIOR ART

Prior art prosthetic hip designs do not incorporate grooves in either cup or ball. The absence of grooves in present art prosthetic hip designs makes it difficult for body fluids to penetrate and lubricate the rubbing surfaces of the cup and ball. (See drawing #8.)

### DRAWING DESCRIPTIONS

Drawing #1 Grooved ball

Drawing #2 Cup without grooves

Drawing #3 Assembly, ball with grooves and cup without grooves

Drawing #4 Ball without grooves

Drawing #5 Cup with grooves

Drawing #6 Assembly, cup with grooves and ball without grooves

Drawing #7 Assembly, ball with grooves and cup with grooves

Drawing #8 Assembly, prior art

Illustration A Spiral groove thrust bearing—Reference 1

### REFERENCES

1. Grooved bearings—Illustration A http://www.tribology-abc.com/calculators/e9_{—}4.htm
2. “Metal Release in Patients Who Have Had a Primary Total Hip Arthroplasty” The Journal of Bone and Joint Surgery, 80:1447-58 (1998) http://www.ejbjs.org/cgi/content/abstract/80/10/1447

1. Prosthetic hip comprising of grooved ball and cup with smooth internal surface (see Drawings #1, #2 and #3).

2. Prosthetic hip comprising of grooved cup and ball without grooves (see Drawings #4, #5 and #6).

3. Prosthetic hip comprising of grooved cup and grooved ball (see drawings #1, #5 and #7).
